(19) United States
(12) Patent Application Publication
(10) Pub. No.: US 2014/0187953 A1
(43) Pub. Date: Jul. 3, 2014

(54) ULTRASOUND DIAGNOSTIC APPARATUS, ULTRASOUND IMAGE PRODUCING METHOD, AND RECORDING MEDIUM

(71) Applicant: FUJIFILM Corporation, Tokyo (JP)

(72) Inventors: Yukiya MIYACHI, Ashigara-kami-gun (JP); Hiroshi YAMAGUCHI, Ashigara-kami-gun (JP); Yasunori OHTA, Ashigara-kami-gun (JP); Tsuyoshi TANABE, Ashigara-kami-gun (JP); Noriaki IDA, Ashigara-kami-gun (JP); Shin NAKATA, Ashigara-kami-gun (JP)

(73) Assignee: FUJIFILM Corporation, Tokyo (JP)

(21) Appl. No.: 14/141,060

(22) Filed: Dec. 26, 2013

(30) Foreign Application Priority Data
Dec. 27, 2012 (JP) 2012-284082

(51) Int. Cl. A61B 8/00; A61B 8/14

(52) U.S. Cl. CPC A61B 8/463; A61B 8/14
USPC 600/447

(57) ABSTRACT

In an ultrasound diagnostic apparatus, when a compound image is produced, delay correction is performed on all of ultrasound images (sub frames) to be combined based on sound velocities set for respective segment regions obtained by dividing the subject. Owing to this configuration, the ultrasound diagnostic apparatus can produce a high quality compound image by spatial compounding or frequency compounding without being affected by distortion in images.

### BACKGROUND OF THE INVENTION

The present invention relates to an ultrasound diagnostic apparatus. In particular, the present invention relates to an ultrasound diagnostic apparatus, an ultrasound image producing method, and a recording medium for use in production of a composite ultrasound image by spatial compounding, frequency compounding, or the like.

Ultrasound diagnostic apparatuses using ultrasound images are put to practical use in the medical field.

In general, this type of ultrasound diagnostic apparatus includes an ultrasound probe (hereinafter also called “probe”) having a piezoelectric element array in which piezoelectric elements transmitting and receiving ultrasonic waves are arranged, and a diagnostic apparatus body.

The ultrasound diagnostic apparatus transmits ultrasonic waves from the probe into a subject's body, receives the ultrasonic echo from the subject with the probe, and electrically processes the resulting reception signals with the diagnostic apparatus body to produce an ultrasound image.

The piezoelectric element array of the ultrasound probe receives through a plurality of piezoelectric elements an ultrasonic echo resulted from one transmission of an ultrasonic beam. Accordingly, even though an ultrasonic echo results from reflection at the same reflection point, the time taken to enter each piezoelectric element varies depending on the position of the piezoelectric element.

To cope with it, the ultrasound diagnostic apparatus performs delay correction separately on reception signals output from the respective piezoelectric elements of the ultrasound probe using a delay time corresponding to, for example, the position of each piezoelectric element, followed by addition (matching addition), thereby producing a proper ultrasound image without distortion.

Meanwhile, so-called “speckle” (speckle noise/speckle pattern) is known as a factor that may deteriorate the image quality of an ultrasound image in the ultrasound diagnostic apparatus. Speckle is white or black spot noise caused by the mutual interference of scattered waves generated by numerous scattering sources which are present in a subject and have a smaller wavelength than that of an ultrasonic wave.

Spatial compounding as described in JP 2005-58321 A is known as a method of reducing such speckle in the ultrasound diagnostic apparatus.

As conceptually shown in FIG. 5, spatial compounding is a technique which involves performing a plurality of types (directions) of transmission and reception of ultrasonic waves in mutually different directions (scanning angles) with respect to a subject by a piezoelectric element unit 100, and combining ultrasound images obtained by the plurality of types of transmission and reception to produce a composite ultrasound image.

Specifically, in the example shown in FIG. 5, three types of transmission and reception of ultrasonic waves (in three directions) are performed which include the transmission and reception of ultrasonic waves as in the normal ultrasound image generation (normal transmission and reception), the transmission and reception of ultrasonic waves in a direction inclined by an angle θ with respect to the direction of the normal transmission and reception, and the transmission and reception of ultrasonic waves in a direction inclined by an angle −θ with respect to the direction of the normal transmission and reception.

An ultrasound image A (solid line) obtained by the normal transmission and reception, an ultrasound image B (broken line) obtained by the transmission and reception in the direction inclined by the angle θ, and an ultrasound image C (dashed-dotted line) obtained by the transmission and reception in the direction inclined by the angle −θ are combined to produce a composite ultrasound image covering the region of the ultrasound image A shown by the solid line.

Alternatively, as a method of reducing such speckle, frequency compounding as described in JP 2000-51210 A is also known.

Frequency compounding is a technique which involves, for instance, performing transmission and reception of ultrasonic waves having a central frequency of f₁ as well as transmission and reception of ultrasonic waves having a different central frequency of f₂, and combining ultrasound images obtained by the two types of transmission and reception to produce a composite ultrasound image.

### SUMMARY OF THE INVENTION

As described above, spatial compounding and frequency compounding enable to produce the high quality ultrasound image with reduced speckle.

On the other hand, when a distorted image is present among ultrasound images to be combined during spatial compounding or frequency compounding, the resulting composite ultrasound image is adversely affected by the distortion, which induces degradation in the image quality.

An object of the present invention is to solve the foregoing problem of the prior art and, as the first aspect, to provide an ultrasound diagnostic apparatus which, when a composite ultrasound image is produced by spatial compounding or frequency compounding, enables to consistently produce a composite ultrasound image with high image quality by producing appropriate ultrasound images to be combined.

An object of the present invention is, as the second aspect, to provide an ultrasound image producing method which, when a composite ultrasound image is produced by spatial compounding or frequency compounding, enables to consistently produce a composite ultrasound image with high image quality by producing appropriate ultrasound images to be combined.

An object of the present invention is, as the third aspect, to provide a recording medium storing a computer program which, when a composite ultrasound image is produced by spatial compounding or frequency compounding, enables to consistently produce a composite ultrasound image with high image quality by producing appropriate ultrasound images to be combined.

In order to attain the object, the present invention provides an ultrasound diagnostic apparatus comprising:

a piezoelectric element array having piezoelectric elements arranged therein, each adapted to transmit ultrasonic waves, receive ultrasonic echoes reflected by a subject, and output reception signals according to received ultrasonic waves;

a controller adapted to control transmission and reception of ultrasonic waves by the piezoelectric element array;

a storage unit adapted to store the reception signals output by the piezoelectric element array;

a sound velocity setting unit adapted to divide the subject into multiple segment regions and set a sound velocity for each of the segment regions with use of the reception signals stored in the storage unit; and

an image producer adapted to produce an ultrasound image by processing the reception signals output by the piezoelectric element array or the reception signals read out from the storage unit based on the sound velocity for each of the segment regions,

wherein the controller has a function of causing the piezoelectric element array to perform transmission and reception for images to be combined to produce images to be combined, the transmission and reception for images to be combined being transmission and reception of ultrasonic waves mutually different in at least one of directions of transmission and reception and central frequencies of ultrasonic waves, and the images to be combined being ultrasound images to be combined;

the image producer has a function of producing the images to be combined with use of the reception signals obtained through the transmission and reception for images to be combined, and combining the produced images to be combined to produce a composite ultrasound image; and

the image producer produces all of the images to be combined based on the sound velocity set for each of the segment regions.

Preferably, in the ultrasound diagnostic apparatus according to the present invention, the sound velocity setting unit sets a sound velocity when an instruction to produce the composite ultrasound image is issued.

Preferably, the image producer produces a main image which is an ultrasound image having a region covering the composite ultrasound image as one of the images to be combined.

Preferably, the sound velocity setting unit sets sound velocities for all of images to be combined.

Preferably, the sound velocity setting unit sets sound velocities only for one image to be combined; and

the image producer produces another image to be combined for which no sound velocities are set based on the sound velocities of corresponding segment regions of the one image to be combined for which the sound velocities are set.

Preferably, the image producer produces a main image which is an ultrasound image having a region covering the composite ultrasound image as one of the images to be combined; and

the one image to be combined for which the sound velocities are set by the sound velocity setting unit is the main image.

Preferably, when the sound velocity setting unit sets sound velocities, the controller causes the piezoelectric element array to perform transmission and reception of ultrasonic waves for sound velocity setting for all of images to be combined;

distortions of reception signals obtained through transmission and reception of ultrasonic waves for the images to be combined are compared among positionally-corresponding segment regions of the images to be combined, and the sound velocity setting unit sets a sound velocity of a corresponding segment region with use of a reception signal having a least distortion; and

the image producer produces the images to be combined based on the sound velocity set for each of the segment regions of all of the images to be combined.

Preferably, the ultrasound diagnostic apparatus has a function of selecting one of the images to be combined for which sound velocities are set by the sound velocity setting unit.

Preferably, the images to be combined are produced from reception signals resulting from transmission and reception of ultrasonic waves for the images to be combined being different in at least directions of transmission and reception of ultrasonic waves; and

the sound velocity setting unit sets sound velocities for an image to be combined being a reference image and sound velocities for another image to be combined resulting from transmission and reception of ultrasonic waves in a direction inclined with respect to a direction of transmission and reception of ultrasonic waves for the reference image by an angle exceeding a predetermined threshold value.

Preferably, an image to be combined for which no sound velocities are set is produced based on sound velocities of corresponding segment regions of an image to be combined for which the sound velocities are set.

Preferably, the controller causes the piezoelectric element array to perform transmission and reception of ultrasonic waves for producing a main image which is an ultrasound image having a region covering the composite ultrasound image as the transmission and reception for images to be combined; and

the direction of transmission and reception of ultrasonic waves for the reference image is a direction of transmission and reception of ultrasonic waves for producing the main image.

The present invention provides an ultrasound image producing method, comprising the steps of:

causing a piezoelectric element array having piezoelectric elements arranged therein, each adapted to transmit ultrasonic waves, receive ultrasonic echoes reflected by a subject, and output reception signals according to received ultrasonic waves, to perform transmission and reception of ultrasonic waves for a plurality of images to be mutually different in either directions of transmission and reception or central frequencies of ultrasonic waves, or both;

setting a sound velocity for each of segment regions obtained by dividing the subject into multiple segment regions with use of reception signals obtained through transmission and reception of ultrasonic waves for each of the plurality of images, and producing a plurality of images to be combined based on the sound velocity set for each of the segment regions, the images to be combined being ultrasound images to be combined, and

combining the images to be combined to produce a composite ultrasound image.

Preferably, in the ultrasound image producing method according to the present invention, the sound velocity is set in response to an instruction to produce the composite ultrasound image.

The present invention provides a recording medium having stored therein a program that causes a computer to implement:

a step of causing a piezoelectric element array having piezoelectric elements arranged therein, each adapted to transmit ultrasonic waves, receive an ultrasonic echo reflected by a subject, and output reception signals according to received ultrasonic waves, to perform transmission and reception of ultrasonic waves being mutually different in at least one of directions of transmission and reception and central frequencies of ultrasonic waves so as to produce a plurality of images;

a step of setting a sound velocity for each of segment regions obtained by dividing the subject into multiple segment regions with use of the reception signals obtained through transmission and reception of ultrasonic waves for each of the plurality of images, and producing a plurality of images to be combined being ultrasound images to be combined based on the sound velocity set for each of the segment regions; and

a step of combining the images to be combined to produce a composite ultrasound image.

Preferably, in the recording medium according to the present invention, the stored program causes the computer to implement a step of setting the sound velocity prior to the step of performing transmission and reception of ultrasonic waves for the plurality of images.

According to the present invention, all of ultrasound images to be combined to produce a composite ultrasound image by spatial compounding or frequency compounding are produced based on appropriate sound velocities. Specifically, all of ultrasound images to be combined to produce a composite ultrasound image are subjected to delay correction with appropriate sound velocities.

Therefore, according to the present invention, it becomes possible to consistently produce a high quality composite ultrasound image by spatial compounding or the like with no degradation in the image quality which may be caused by distortion in ultrasound images to be combined.

### BRIEF DESCRIPTION OF THE DRAWINGS

FIG. 1 is a block diagram conceptually showing an ultrasound diagnostic apparatus of the invention.

FIGS. 2A to 2C are conceptual diagrams for explaining spatial compounding performed in the ultrasound diagnostic apparatus shown in FIG. 1.

FIGS. 3A to 3C are conceptual diagrams for explaining frequency compounding performed in the ultrasound diagnostic apparatus shown in FIG. 1.

FIGS. 4A and 4B are conceptual diagram for explaining an example of a sound velocity setting method in the ultrasound diagnostic apparatus shown in FIG. 1.

FIG. 5 is a conceptual diagram for explaining spatial compounding.

### DETAILED DESCRIPTION OF THE INVENTION

An ultrasound diagnostic apparatus, an ultrasound image producing method, and a recording medium of the invention will be described in detail below with reference to the preferred embodiments shown in the accompanying drawings.

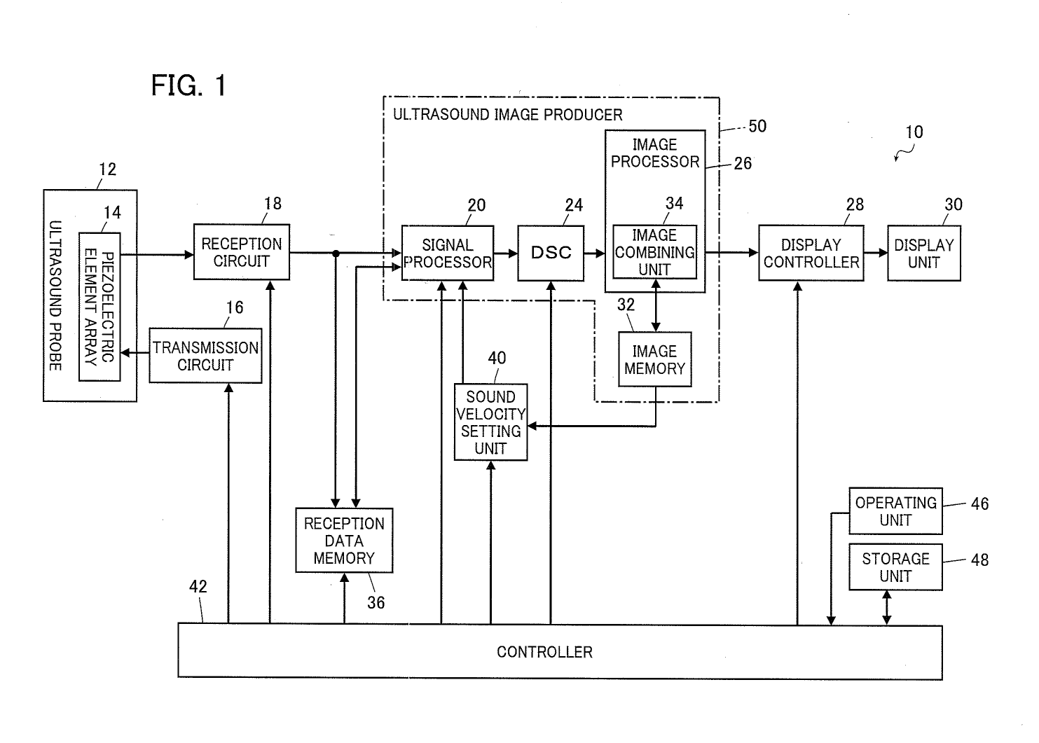

FIG. 1 is a block diagram conceptually showing an example of an ultrasound diagnostic apparatus of the invention which performs an ultrasound image producing method of the invention.

As shown in FIG. 1, an ultrasound diagnostic apparatus 10 has an ultrasound probe 12 (hereinafter called “probe 12”) including a piezoelectric element array 14.

The piezoelectric element array 14 of the probe 12 is connected to a transmission circuit 16 and a reception circuit 18. The reception circuit 18 is connected in sequence to a signal processor 20, a digital scan converter (DSC) 24, an image processor 26, a display controller 28, and a display unit 30. The image processor 26 is connected to an image memory 32. In addition, the image processor 26 includes an image combining unit 34.

The signal processor 20, the DSC 24, the image processor 26, and the image memory 32 constitute an image producer 50.

The reception circuit 18 and the signal processor 20 are connected to a reception data memory 36, and the image memory 32 and the signal processor 20 are connected to a sound velocity setting unit 40.

Furthermore, the transmission circuit 16, the reception circuit 18, the signal processor 20, the DSC 24, the display controller 28, the reception data memory 36, and the sound velocity setting unit 40 are connected to a controller 42. The controller 42 is also connected to an operating unit 46 and a storage unit 48.

In the illustrated example, the transmission circuit 16, the reception circuit 18, the ultrasound image producer 50, the display controller 28, the display unit 30, the reception data memory 36, the sound velocity setting unit 40, the controller 42, the operating unit 46, and the storage unit 48 constitute a diagnostic apparatus body of the ultrasound diagnostic apparatus 10.

The diagnostic apparatus body is constituted by, for example, a computer.

The piezoelectric element array 14 includes a plurality of piezoelectric elements (ultrasound transducers) arranged one-dimensionally or two-dimensionally. These piezoelectric elements each transmit ultrasonic waves according to driving signals supplied from the transmission circuit 16 and receive ultrasonic echoes from the subject to output reception signals.

The piezoelectric element is composed of a vibrator in which electrodes are provided at the both ends of a piezoelectric body. The piezoelectric body may be composed of, for example, a piezoelectric ceramic typified by lead zirconate titanate (PZT), a piezoelectric polymer typified by polyvinylidene fluoride (PVDF), or a piezoelectric monocrystal typified by lead magnesium niobate-lead titanate solid solution (PMN-PT).

When a pulsed voltage or a continuous-wave voltage is applied to the electrodes of such a vibrator, the piezoelectric body expands and contracts to cause the vibrator to generate pulsed or continuous ultrasonic waves. These ultrasonic waves are synthesized to form an ultrasonic beam.

Upon reception of propagating ultrasonic waves, the vibrators expand and contract to produce electric signals. The electric signals are output from the piezoelectric elements (piezoelectric element array 14) as reception signals of the ultrasonic waves.

The transmission circuit 16 includes, for instance, a plurality of pulse generators. The transmission circuit 16 adjusts delay amounts of the driving signals and then supplies the adjusted driving signals to the respective piezoelectric elements so that the ultrasonic waves transmitted from the piezoelectric element array 14 form an ultrasonic beam as desired. The transmission circuit 16 adjusts each delay amount based on a transmission delay pattern selected in accordance with a control signal from the controller 42.

The reception circuit 18 amplifies the reception signals transmitted from the piezoelectric elements of the piezoelectric element array 14 and analog-to-digital converts the amplified signals to produce pieces of digitalized reception data as many as the number of reception channels.

The ultrasound diagnostic apparatus 10 has the function of performing spatial compounding.

As known, spatial compounding is a method of producing an ultrasound image with reduced speckle by combining a plurality of ultrasound images obtained by transmission and reception of ultrasonic waves in mutually different directions to produce a composite ultrasound image. In the following explanation, a composite ultrasound image obtained by spatial compounding or frequency compounding to be described below is also referred to as a compound image.

When spatial compounding is performed, the transmission circuit 16 and the reception circuit 18 cause the piezoelectric element array 14 in accordance with an instruction by the controller 42 to perform multiple times of transmission and reception of ultrasonic waves in mutually different directions of transmission and reception of ultrasonic waves. In spatial compounding, the multiple times of transmission and reception of ultrasonic waves are performed to produce a plurality of images to be combined (ultrasound images to be combined) obtained by transmission and reception of ultrasonic waves in mutually different directions to produce a composite ultrasound image (compound image).

For convenience, the “transmission and reception of ultrasonic waves” is also referred to simply as “transmission and reception.”

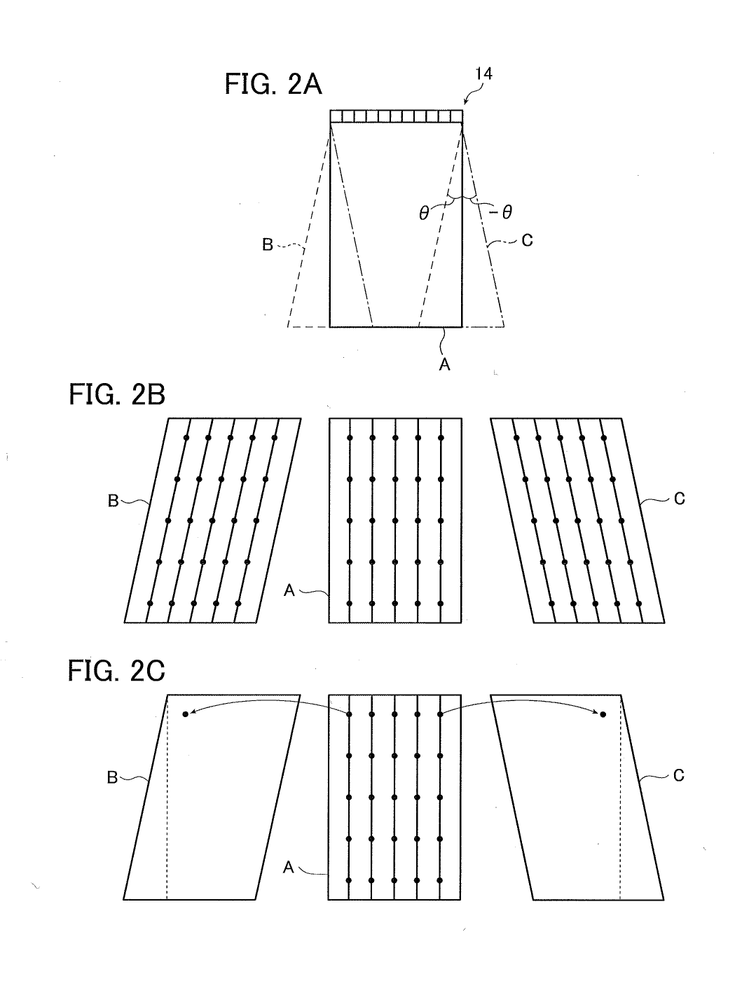

As an example, the ultrasound diagnostic apparatus 10 combines three images by spatial compounding. Specifically, as conceptually shown in FIG. 2A, the piezoelectric element array 14 performs three types of transmission and reception which include transmission and reception in the same direction as in the normal ultrasound image generation (see the solid line), transmission and reception in a direction inclined by an angle θ with respect to the direction of the transmission and reception for the normal ultrasound image generation (see the broken line), and transmission and reception in a direction inclined by an angle −θ with respect to the direction of the transmission and reception for the normal ultrasound image generation (see the dashed-dotted line).

When spatial compounding with three images to be combined is performed, three types of transmission and reception are performed to produce one (one frame of) compound image. Accordingly, when spatial compounding with three images to be combined is performed, the piezoelectric element array 14 repeatedly performs the three types of transmission and reception.

Specifically, in the exemplary spatial compounding shown in FIG. 2, there are produced an image to be combined A (sub frame A) indicated by the solid line and obtained by transmission and reception in the same direction as in the normal ultrasound image generation; an image to be combined B (sub frame B) indicated by the broken line and obtained by transmission and reception in a direction inclined by the angle θ with respect to the direction of the transmission and reception for the image to be combined A; and an image to be combined C (sub frame C) indicated by the dashed-dotted line and obtained by transmission and reception in a direction inclined by the angle −θ with respect to the direction of the transmission and reception for the image to be combined A.

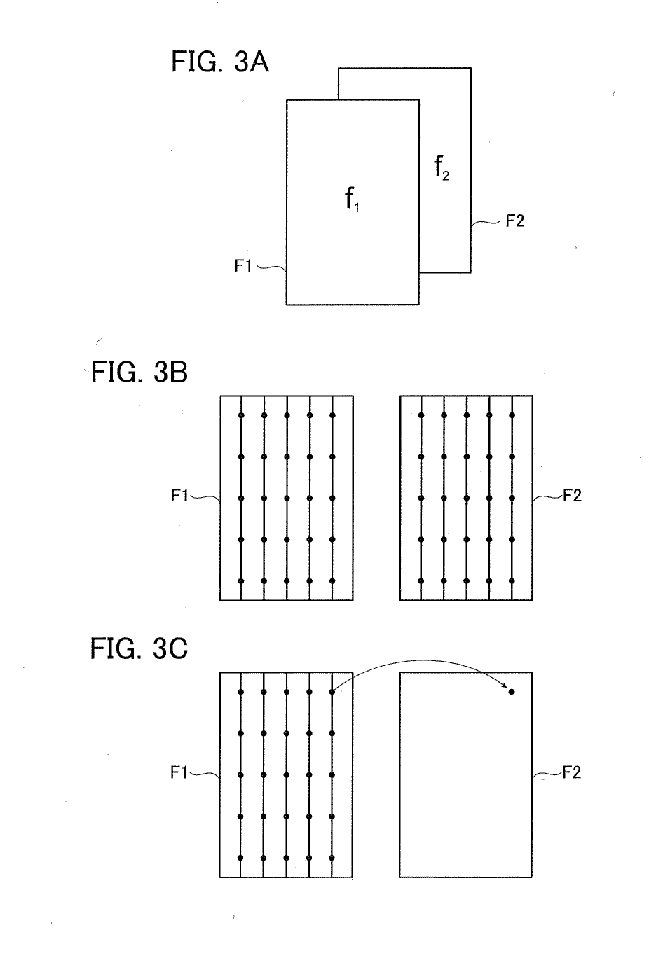

For convenience, the transmission and reception for producing the image to be combined A is also referred to as transmission and reception for image A, the transmission and reception for producing the image to be combined B as transmission and reception for image B, and the transmission and reception for producing the image to be combined C as transmission and reception for image C. In this regard, the same applies to an alternative embodiment shown in FIG. 3 or the like to be described below.

The image combining unit 34 of the image processor 26 to be described below combines the image to be combined A, the image to be combined B and the image to be combined C to produce a compound image having the same region as that of the image to be combined A which is equivalent to an image obtained by the normal transmission and reception. Specifically, the image to be combined A is a main image (basic image/basic sub frame) in this spatial compounding.

It should be noted that, in the present invention, the number of images to be combined by spatial compounding may be two, four or more.

The signal processor 20 implements delay correction on each piece of reception data produced by the reception circuit 18 based on a sound velocity (a set sound velocity and an optimal sound velocity to be described below) input from the sound velocity setting unit 40. The signal processor 20 produces pieces of delay correction data through the delay processing and adds those pieces of delay correction data (performs matching addition) to perform a reception focusing process. By this process, the ultrasonic echo is well focused so as to produce a sound ray signal. Furthermore, the signal processor 20 corrects the sound ray signal for the attenuation due to distance according to the depth at which the ultrasonic waves are reflected. The signal processor 20 further implements an envelope detection process on the sound ray signal having been subjected to attenuation correction to thereby produce a B-mode image signal which is tomographic image information relating to the tissue in the subject.

The DSC 24 converts the B-mode image signal produced by the signal processor 20 into an image signal compatible with an ordinary television signal scanning mode (raster conversion).

The image processor 26 performs various kinds of necessary image processing such as gradation processing on the B-mode image signal entered from the DSC 24, and outputs the B-mode image signal to the display controller 28. Alternatively, after subjecting the B-mode image signal entered from the DSC 24 to necessary image processing, the image processor 26 stores the B-mode image signal in the image memory 32.

As described above, the ultrasound image producer 50 is made up of the signal processor 20, the DSC 24, the image processor 26, and the image memory 32.

The image processor 26 includes the image combining unit 34.

The image combining unit 34 combines produced images to be combined to produce a compound image when spatial compounding or frequency compounding to be described below is performed.

The display controller 28 causes the monitor 30 to display an ultrasound diagnostic image and the like according to the B mode image signal having undergone image processing by the image processor 26 or various types of information input by the operating unit 46.

The display unit 30 includes a display device such as an LCD, for example, and displays the ultrasound diagnostic image under the control of the display controller 28.

The reception data memory 36 sequentially stores the reception data output from the reception circuit 18 and also stores the delay correction data produced by the signal processor 20.

The sound velocity setting unit 40 sets optimal sound velocities that are sound velocities of (the inside of) the subject.

In the present invention, the sound velocity setting unit 40 divides the inside of the subject into multiple segment regions and sets the optimal sound velocity for each of the segment regions. As an example, the sound velocity setting unit 40 provides a predetermined set sound velocity to the signal processor 20, while changing the set sound velocity, causes the signal processor 20 to produce sound ray signals, and causes the ultrasound image producer 50 to produce B-mode image signals with the use of the sound ray signals obtained based on the respective set sound velocities. The sound velocity setting unit 40 analyzes B-mode images thus produced with the different set sound velocities and sets a sound velocity at which the contrast or the sharpness of the image is highest as the optimal sound velocity of each segment region of the subject.

The controller 42 controls components of the ultrasound diagnostic apparatus according to instructions entered by the operator using the operating unit 46.

The operating unit 46 is provided for the operator to perform input operations and may be composed of, for example, a keyboard, a mouse, a track ball, and/or a touch panel.

The storage unit 48 stores, for example, an operation program and may be constituted by, for example, a recording medium such as a hard disk, a flexible disk, an MO, an MT, a RAM, a CD-ROM, a DVD-ROM, an SD card, a CF card, and a USB memory, or a server.

The signal processor 20, the DSC 24, the image processor 26, the display controller 28, and the sound velocity setting unit 40 are each constituted by a CPU and an operation program for causing the CPU to perform various kinds of processing, but they may be each constituted by a digital circuit.

The present invention will be explained in further detail by explaining the operation of spatial compounding in the ultrasound diagnostic apparatus 10. A recording medium according to the present invention is a recording medium that has a program stored therein for causing a computer to implement the ultrasound image producing method of the invention to be described below and is readable by a computer (the same applies to the case of frequency compounding to be described below).

As described above, in the ultrasound diagnostic apparatus 10, the subject is divided into multiple segment regions and is set with optimal sound velocities which are sound velocities of the respective segment regions.

In addition, in this example, optimal sound velocities are set to separately correspond to the respective images to be combined A, B and C to be combined by spatial compounding.

In the ultrasound diagnostic apparatus 10, setting (resetting/updating) of sound velocities is performed at appropriately-set predetermined timing.

Various kinds of timing can be applied for setting optimal sound velocities. For example, an optimal sound velocity may be set at the start of a diagnosis, set for every predetermined number of frames, set when the probe 12 has been moved by a predetermined distance or more, or set when the probe 12 has remained at one place for a predetermined period of time or more. In this invention, it is preferred to set (update) an optimal sound velocity when an instruction to perform spatial compounding is issued (the same applies to the case of frequency compounding to be described below).

When optimal sound velocities are set, the controller 42 sends instructions to the transmission circuit 16 and the reception circuit 18 to cause the piezoelectric element array 14 to perform transmission and reception for sound velocity setting.

In this example, in transmission and reception for producing an ultrasound image, transmission and reception having a predetermined focal point is performed one time for one sound ray signal to be produced (for a certain position in the azimuth direction). Alternatively, in this example, in transmission and reception for producing an ultrasound image, transmission and reception is performed two times with different focal points (focal points different in position in the depth direction) for one sound ray signal to be produced.

On the other hand, in transmission and reception for sound velocity setting in the ultrasound diagnostic apparatus 10, transmission and reception is performed more times than the transmission and reception for producing the normal ultrasound image with mutually different focal points for one sound ray signal to be produced. In addition, the number of sound ray signals (the density of sound rays in the azimuth direction) may also be increased more than that in the transmission and reception for producing an ultrasound image.

FIG. 2B conceptually illustrates an example of the transmission and reception for sound velocity setting.

In this example, transmission and reception is performed five times for one sound ray signal to set the optimal sound velocity. In FIG. 2B, the solid lines in the images to be combined represent sound ray signals (i.e., scanning lines to be produced). The points on the sound ray signals represent focal points of transmitted ultrasonic beams. Specifically, in this transmission and reception for sound velocity setting, transmission of an ultrasonic beam is performed five times with mutually different focal points to produce one sound ray signal.

In the ultrasound diagnostic apparatus 10, when optimal sound velocities of the subject are set, transmission and reception for sound velocity setting in association with the image to be combined A is first performed as shown in FIG. 2B.

In the transmission and reception for sound velocity setting, reception signals output from the respective piezoelectric elements of the piezoelectric element array 14 undergo amplification and A/D conversion by the reception circuit 18, and the resulting pieces of reception data are sequentially stored in the reception data memory 36.

At the same time, the sound velocity setting unit 40 supplies a first set sound velocity S1 to the signal processor 20.

The signal processor 20 reads out the pieces of reception data stored in the reception data memory 36, implements delay correction on the pieces of reception data based on the supplied first set sound velocity S1 to produce pieces of delay data, and adds the produced pieces of delay data to perform the reception focusing process to thereby produce a sound ray signal. The signal processor 20 further implements the correction of attenuation and the envelope detection process on the sound ray signal to thereby produce a B-mode image signal.

The B-mode image signal undergoes raster conversion by the DSC 24 and then various kinds of image processing by the image processor 26, and subsequently, is stored in the image memory 32 as a B-mode image signal for sound velocity setting in association with the image to be combined A.

Upon storage of the B-mode image signal corresponding to the first set sound velocity S1 supplied from the sound velocity setting unit 40 into the image memory 32, the sound velocity setting unit 40 supplies the signal processor 20 with a second set sound velocity S2 having a value changed from the first set sound velocity S1 by a predetermined amount.

The sound velocity setting unit 40 thus provides a plurality of set sound velocities S1 to Sn to the signal processor 20 in sequence, and B-mode image signals corresponding to those set sound velocities S1 to Sn are produced by the ultrasound image producer 50 and stored in the image memory 32. After the B-mode image signals corresponding to the set sound velocities S1 to Sn are stored in the image memory 32, the sound velocity setting unit 40 performs the analysis on the B-mode image signals stored in the image memory 32. Based on the results of the analysis, the sound velocity setting unit 40 sets a sound velocity at which the contrast or the sharpness of the image is highest as the optimal sound velocity for the image to be combined A.

Specifically, the optimal sound velocity is a sound velocity from a certain predetermined position (a segment region to be described below) to the piezoelectric elements, where the sound velocity is considered as constant in the subject from the predetermined position to the piezoelectric elements. In other words, the optimal sound velocity is an average sound velocity in the subject from a certain predetermined position (a segment region to be described below) to the piezoelectric elements.

In setting optimal sound velocities, the analysis of the B-mode image signals is performed for each of segment regions obtained by dividing the subject (ultrasound image), and an optimal sound velocity is set for each of the segment regions. Specifically, a sound velocity at which the contrast or the sharpness of the image is highest is selected to be set as the optimal sound velocity for each of the segment regions.

In the illustrated example, as an example, the image to be combined is divided to establish a grid pattern (by dividing in the azimuth direction and in the direction parallel to a direction of transmission of ultrasonic beams) with focal points of ultrasonic beams being taken as centers of the respective segment regions, and an optimal sound velocity is set for each of the thus-obtained segment regions. Specifically, an optimal sound velocity is set to correspond to each focal point that is formed in the transmission and reception for sound velocity setting.

It should be noted that division of the subject for which optimal sound velocities are set, i.e., focal points formed in the transmission and reception for sound velocity setting, may be suitably set in accordance with, for instance, required accuracy of tissue elasticity measurement, required image quality, or required processing speed.

Preferably, focal points are each formed at the same position in every pixel of an ultrasound image to be produced. Alternatively, one focal point may be given for several pixels whose number is appropriately determined in such a manner of giving, for example, one focal point per three pixels, nine pixels, and so forth. Still alternatively, segment regions may be set by equally dividing an ultrasound image by an appropriately-set number, for example, by 10 or 20.

Furthermore, the number of segment regions, the number of focal points on one scanning line, or the like may be determined by the operator. The foregoing setting of segment regions may be made through the operation of mode selection or the like.

After the optimal sound velocities for the image to be combined A are set, then the ultrasound diagnostic apparatus 10 performs transmission and reception for sound velocity setting in association with the image to be combined B as shown in FIG. 2B to set optimal sound velocities of respective segment regions for the image to be combined B in the same manner as the image to be combined A.

In addition, after the optimal sound velocities for the image to be combined B are set, then the ultrasound diagnostic apparatus 10 performs transmission and reception for sound velocity setting in association with the image to be combined C as shown in FIG. 2B to set optimal sound velocities of respective segment regions for the image to be combined C in the same manner as the image to be combined A.

The optimal sound velocities of the respective segment regions of the images to be combined A, B and C set by the sound velocity setting unit 40 are each linked with a relevant image to be combined and a relevant segment region, and then supplied to the signal processor 20 to be stored therein. Alternatively, the optimal sound velocities may be each linked with a relevant image to be combined and a relevant segment region, and then stored in the storage unit 48, so that the controller 42 reads out the optimal sound velocities to supply them to the signal processor 20.

A method of setting a sound velocity of a subject is not limited to the foregoing method and use may be made of various known sound velocity setting methods employed in ultrasound diagnostic apparatuses or ultrasound image generating methods.

In the ultrasound diagnostic apparatus of the invention, a compound image for use in display may be produced by using reception data acquired through transmission and reception for updating sound velocities. Alternatively, reception data acquired through transmission and reception for updating sound velocities may be processed based on the optimal sound velocities having been set (updated) with the use of this reception data, thereby producing a compound image for use in display. When a compound image (image to be combined) is produced with the use of reception data acquired through transmission and reception for updating sound velocities, processing such as thinning may be performed as necessary.

When optimal sound velocities are set at timing not corresponding to the instruction to perform spatial compounding, optimal sound velocities may be set only for the image to be combined A.

In the ultrasound diagnostic apparatus 10, when an ultrasound image (compound image (composite ultrasound image)) is produced by spatial compounding, in response to the instruction from the controller 42, the transmission circuit 16 and the reception circuit 18 cause the piezoelectric element array 14 to sequentially perform the transmission and reception for image A, the transmission and reception for image B, and then the transmission and reception for image C.

Reception signals output from the piezoelectric element array 14 through the transmission and reception are processed by the reception circuit 18 and the resulting reception data is supplied to the signal processor 20. Alternatively, the reception data may be stored in the reception data memory 36 as necessary. Still alternatively, the signal processor 20 may read out the reception data from the reception data memory 36 and subject the reception data to processing below.

The signal processor 20 having acquired the reception data performs dedicated delay correction on each piece of the reception data based on the stored optimal sound velocities, thereby producing pieces of delay correction data.

At this time, the signal processor 20 has stored therein the optimal sound velocities of the respective segment regions for the images to be combined A, B and C with each optimal sound velocity being linked with a relevant image to be combined and a relevant segment region.

Accordingly, for the reception data resulting from the transmission and reception for image A, the signal processor 20 produces delay correction data based on the optimal sound velocities of corresponding segment regions as set for the image to be combined A. For the reception data resulting from the transmission and reception for image B, the signal processor 20 produces delay correction data based on the optimal sound velocities of corresponding segment regions as set for the image to be combined B. For the reception data resulting from the transmission and reception for image C, the signal processor 20 produces delay correction data based on the optimal sound velocities of corresponding segment regions as set for the image to be combined C.

Subsequently, the signal processor 20 adds the produced pieces of delay correction data (performs matching addition) to perform the reception focusing process to thereby produce a sound ray signal. In addition, the signal processor 20 performs the correction of attenuation and the envelope detection process on the produced sound ray signal.

As a result, B-mode image signals of the images to be combined A, B and C are produced.

The B-mode image signals of the images to be combined A, B and C (hereinafter the phrase “B-mode image signals of” is omitted) undergo raster conversion by the DSC 24 and predetermined image processing by the image processor 26.

Then the images to be combined A, B and C are combined by the image combining unit 34 of the image processor 26 so that a compound image (B-mode image signal thereof) having the same region as the region of the image to be combined A is produced.

The produced compound image is supplied to the display controller 28 and displayed on the display unit 30. In addition, the produced compound image is stored in the image memory 32 as necessary.

This compound image is an image in which all of the images to be combined A, B and C have been subjected to delay correction (sound velocity correction) in accordance with the optimal sound velocity as set for each of the segment regions. Therefore, it becomes possible to greatly suppress image degradation which may be caused by distortion in the images to be combined to thereby produce a high quality compound image.

In the foregoing embodiment, optimal sound velocities are set for the respective segment regions in association with all of the images to be combined.

On the other hand, in an alternative embodiment of the invention, optimal sound velocities are set for the respective segment regions in association with only one of the images to be combined. In this embodiment, images to be combined for which no optimal sound velocities are set are subjected to delay correction with the use of the optimal sound velocities of corresponding segment regions of one image to be combined for which the optimal sound velocities are set, so as to produce a compound image.

Even when this method of producing a compound image is applied, similarly it becomes possible to greatly suppress image degradation which may be caused by distortion in the images to be combined to thereby produce a high quality compound image.

An example of this embodiment is conceptually shown in FIG. 2C.

In this example, as a preferred embodiment, optimal sound velocities of respective segment regions are set only for the image to be combined A which is the main image in spatial compounding. Specifically, in this example, optimal sound velocities of respective segment regions are set only for the image to be combined A having (or including) the same region as that of a compound image to be produced.

Accordingly, when optimal sound velocities are set, the transmission circuit 16 and the reception circuit 18 cause the piezoelectric element array 14 to perform the transmission and reception for sound velocity setting only for the image to be combined A, as shown in FIG. 2C.

Reception signals output from the piezoelectric element array 14 are processed by the reception circuit 18 to be reception data, and the reception data is stored in the reception data memory 36. Similarly to the foregoing, the sound velocity setting unit 40 supplies the set sound velocities S1 to Sn to the signal processor 20. The signal processor 20 produces delay data with the use of the supplied set sound velocities S1 to Sn to produce sound ray signals. In response, the ultrasound image producer 50 produces B-mode image signals through the transmission and reception for sound velocity setting relevant to the image to be combined A. The sound velocity setting unit 40 sets the optimal sound velocities for each of the segment regions in association with the image to be combined A, and supplies the set optimal sound velocities to the signal processor 20 to be stored therein.

When spatial compounding is performed, similarly to the foregoing, the transmission circuit 16 and the reception circuit 18 cause the piezoelectric element array 14 to sequentially perform the transmission and reception for image A, the transmission and reception for image B, and then the transmission and reception for image C.

Reception signals output from the piezoelectric element array 14 through the transmission and reception are processed by the reception circuit 18 and the resulting reception data is supplied to the signal processor 20.

The signal processor 20 having acquired the reception data performs delay correction on reception data resulting from the transmission and reception for image A based on the optimal sound velocities of corresponding segment regions as set for the image to be combined A, thereby producing delay correction data.

As for reception data resulting from the transmission and reception for image B, as conceptually shown in FIG. 2C, the optimal sound velocities of corresponding segment regions as set for the image to be combined A are used to produce delay correction data for respective segment regions of the image to be combined B. Similarly, also for reception data resulting from the transmission and reception for image C, the optimal sound velocities of corresponding segment regions as set for the image to be combined A are used to produce delay correction data for respective segment regions of the image to be combined C.

The broken lines in the images to be combined B and C in FIG. 2C represent edges of the image to be combined A in the azimuth direction when the image to be combined A overlaps the images to be combined B and C.

Subsequently, similarly to the foregoing, the signal processor 20 adds the produced pieces of delay correction data to produce a sound ray signal, and performs the correction of attenuation and the envelope detection process on the produced sound ray signal, thereby producing the images to be combined A, B and C (B-mode images thereof).

The images to be combined A, B and C undergo raster conversion by the DSC 24 and predetermined image processing by the image processor 26, and the images to be combined A, B and C are combined by the image combining unit 34 to produce a compound image.

The compound image produced by the ultrasound image producer 50 is transmitted to the display controller 28 and then to the display unit 30 to be displayed on the display unit 30.

In the present invention, as an alternative embodiment, of images to be combined, images to be set with optimal sound velocities may be selected.

Specifically, among images to be combined which greatly differ in the inclination angle of associated transmission and reception, the transmission routes of ultrasonic beams and ultrasonic echoes greatly differ among those images. Accordingly, among images to be combined which greatly differ in the inclination angle of associated transmission and reception, optimal sound velocities of corresponding segment regions may be notably differ among those images.

To cope with it, optimal sound velocities may be set in such a manner that an image to be combined is selected as a reference image, an angle between a direction of transmission and reception for the reference image to be combined and a direction of transmission and reception for the other images to be combined is detected, and optimal sound velocities are set for, in addition to the reference image to be combined, an image or images with the detected angle exceeding a threshold value.

As an example, in FIG. 2, as a preferred embodiment, the image to be combined A which is the main image is set as the reference image. Accordingly, the image to be combined A is set with optimal sound velocities.

With regard to the images to be combined B and C, when the absolute value of the angle θ or the angle −θ is less than a predetermined threshold value, no optimal sound velocities are set and the optimal sound velocities of corresponding segment regions of the image to be combined A are used to produce delay correction data for the images to be combined B and C. In contrast, when the absolute value of the angle θ or the angle −θ is equal to or greater than the predetermined threshold value, optimal sound velocities are set for the images to be combined B and C as well.

Alternatively, in the case of combining five images of, in addition to the images to be combined A, B and C, other two images to be combined D and E in which directions of their transmission and reception are inclined by an angle η or an angle −η greater in the absolute value than the angle θ or the angle −θ, optimal sound velocities may be set in the same manner. Specifically, also in this case, the image to be combined A being the main image is set with optimal sound velocities similarly to the foregoing. When the absolute value of the angle θ or the angle −θ is less than the predetermined threshold value, no optimal sound velocities are set for the images to be combined B and C. In contrast, when the absolute value of the angle η or the angle −η is equal to or greater than the predetermined threshold value, optimal sound velocities are set for the images to be combined D and E.

In spatial compounding with five images as in the above case, when the absolute value of the angle θ or the angle −θ and the absolute value of the angle η or the angle −η are all less than the threshold value, the image to be combined A being the reference image is solely set with optimal sound velocities, and no optimal sound velocities are set for the other images to be combined. In contrast, when the absolute value of the angle θ or the angle −θ and the absolute value of the angle η or the angle −η are all equal to or greater than the threshold value, optimal sound velocities are set for all the images to be combined.

In this example, when spatial compounding is performed, for the image to be combined for which optimal sound velocities are set, delay correction of reception data is performed based on the set optimal sound velocities to produce the image to be combined, as in the above example. On the other hand, for the image to be combined for which no optimal sound velocities are set, delay correction is performed based on the optimal sound velocities of corresponding segment regions as set for the image to be combined for which the optimal sound velocities are set, thereby producing the image to be combined.

In the following processing, similarly to the foregoing, the images to be combined are processed by the DSC 24 and the image processor 26 and combined by the image combining unit 34 to produce a compound image, and the produced compound image is displayed on the display unit 30.

The ultrasound diagnostic apparatus 10 shown in FIG. 1 may have, in addition to (or instead of) the function of performing spatial compounding, the function of performing frequency compounding. The ultrasound diagnostic apparatus 10 may have the function of simultaneously performing both spatial compounding and frequency compounding.

As known, frequency compounding is a method of producing a compound image (composite ultrasound image) by combining a plurality of ultrasound images to be combined obtained through transmission and reception of ultrasonic waves having mutually different central frequencies.

Hence, when frequency compounding is performed, the controller 42 issues an instruction to the transmission circuit 16 and the reception circuit 18 to cause the piezoelectric element array 14 to perform transmission and reception of ultrasonic waves having mutually different central frequencies for a plurality of images.

For instance, as shown in FIG. 3A, in frequency compounding, a compound image is produced by combining an image to be combined F1 obtained through transmission and reception with a central frequency f₁ and an image to be combined F2 obtained through transmission and reception with a central frequency f₂ (f₁<f₂).

In this case, optimal sound velocities may be set for all the images to be combined as shown in FIG. 2B described above. Alternatively, in this case, optimal sound velocities may be set only for one image to be combined as shown in FIG. 2C.

As an example, when optimal sound velocities are set, as conceptually shown in FIG. 3B, the transmission circuit 16 and the reception circuit 18 cause the piezoelectric element array 14 to perform the transmission and reception for sound velocity setting using ultrasonic waves having the central frequency f₁ and the transmission and reception for sound velocity setting using ultrasonic waves having the central frequency f₂ for the images to be combined F1 and F2.

Reception signals output from the piezoelectric element array 14 are processed by the reception circuit 18 to be reception data, and the reception data is stored in the reception data memory 36. In addition, similarly to the foregoing, the sound velocity setting unit 40 supplies the set sound velocities S1 to Sn to the signal processor 20. The signal processor 20 produces delay data with the use of the supplied set sound velocities S1 to Sn to produce sound ray signals. In response, the ultrasound image producer 50 produces B-mode image signals resulting from the transmission and reception for sound velocity setting for the images to be combined F1 and F2. Further, similarly to the foregoing, the sound velocity setting unit 40 sets optimal sound velocities of respective segment regions for the images to be combined F1 and F2, and supplies the set optimal sound velocities to the signal processor 20. The signal processor 20 links each of the optimal sound velocities with a relevant image to be combined and a relevant segment region and stores the optimal sound velocities.

When frequency compounding is performed, the transmission circuit 16 and the reception circuit 18 cause the piezoelectric element array 14 to sequentially perform the transmission and reception of ultrasonic waves having the central frequency f₁ for the image F1 and the transmission and reception of ultrasonic waves having the central frequency f₂ for the image F2.

Reception signals output from the piezoelectric element array 14 in response to the transmission and reception are processed by the reception circuit 18 to be reception data, and the reception data is supplied to the signal processor 20.

The signal processor 20 having acquired the reception data performs delay correction on the reception data resulting from the transmission and reception for the image F1 based on the optimal sound velocities of corresponding segment regions as set for the image to be combined F1, thereby producing delay correction data. Also, the signal processor 20 performs delay correction on the reception data resulting from the transmission and reception for the image F2 based on the optimal sound velocities of corresponding segment regions as set for the image to be combined F2, thereby producing delay correction data.

Subsequently, similarly to the foregoing, the signal processor 20 adds the produced pieces of delay correction data to perform the reception focusing process, produces a sound ray signal, and performs the correction of attenuation and the envelope detection process on the produced sound ray signal to produce the images to be combined F1 and F2 (B-mode images thereof).

The respective images to be combined undergo raster conversion by the DSC 24 and predetermined image processing by the image processor 26, and the images to be combined F1 and F2 are combined by the image combining unit 34 to produce a compound image.

The compound image produced by the ultrasound image producer 50 is transmitted to the display controller 28 and then to the display unit 30 to be displayed on the display unit 30.

In another embodiment of the invention, when frequency compounding is performed, similarly to the foregoing, optimal sound velocities may be set only for one image to be combined and the other image to be combined may utilize the optimal sound velocities of corresponding segment regions of the image to be combined for which the optimal sound velocities are set.

Also in this case, preferably optimal sound velocities are set for the main image. The main image in the case of frequency compounding is an image to be combined obtained through transmission and reception of ultrasonic waves having the same central frequency as that in production of the normal ultrasound image.

In this case, when optimal sound velocities are set, as conceptually shown in FIG. 3C, the transmission circuit 16 and the reception circuit 18 cause the piezoelectric element array 14 to perform transmission and reception for sound velocity setting using ultrasonic waves having the central frequency f₁ only for the image to be combined F1.

Reception signals output from the piezoelectric element array 14 are processed by the reception circuit 18 to be reception data, and the reception data is stored in the reception data memory 36. Similarly to the foregoing, the sound velocity setting unit 40 supplies the set sound velocities S1 to Sn to the signal processor 20. The signal processor 20 produces delay data with the use of the supplied set sound velocities S1 to Sn to produce sound ray signals. In response, the ultrasound image producer 50 produces B-mode image signals resulting from the transmission and reception for sound velocity setting for the image to be combined F1. Further, similarly to the foregoing, the sound velocity setting unit 40 sets optimal sound velocities of respective segment regions for the image to be combined F1. The signal processor 20 links each of the optimal sound velocities with a relevant segment region of the image to be combined F1 and stores the optimal sound velocities.

When frequency compounding is performed, the transmission circuit 16 and the reception circuit 18 cause the piezoelectric element array 14 to sequentially perform the transmission and reception of ultrasonic waves having the central frequency f₁ for the image F1 and the transmission and reception of ultrasonic waves having the central frequency f₂ for the image F2.

Reception signals output from the piezoelectric element array 14 through the transmission and reception are processed by the reception circuit 18 to be reception data, and the reception data is supplied to the signal processor 20.

The signal processor 20 having acquired the reception data performs delay correction on the reception data resulting from the transmission and reception for the image F1 based on the optimal sound velocities of corresponding segment regions as set for the image to be combined F1, thereby producing delay correction data.

On the other hand, for the reception data produced through the transmission and reception for the image F2, as conceptually shown in FIG. 3C, the optimal sound velocities of corresponding segment regions as set for the image to be combined F1 are used to produce delay correction data for respective segment regions of the image to be combined F2.

Subsequently, similarly to the foregoing, the signal processor 20 adds the produced pieces of delay correction data to perform the reception focusing process, produces a sound ray signal, and performs the correction of attenuation and the envelope detection process on the produced sound ray signal, thereby producing the images to be combined F1 and F2.

The respective images to be combined undergo raster conversion by the DSC 24 and predetermined image processing by the image processor 26, and the images to be combined F1 and F2 are combined by the image combining unit 34 to produce a compound image.

The compound image produced by the ultrasound image producer 50 is transmitted to the display controller 28 and then to the display unit 30 to be displayed on the display unit 30.

In the present invention, the number of images to be combined by frequency compounding may be three or more.

Furthermore, frequency compounding in the present invention includes the case of combining images to be combined obtained by transmission and reception of ultrasonic waves having the same central frequencies and an image or images to be combined obtained by so-called harmonic imaging. As known, harmonic imaging is used in production of an ultrasound image by receiving, for example, second harmonic of ultrasonic echoes with respect to the frequency of transmitted ultrasonic waves. In this case, central frequencies of ultrasonic waves transmitted for images to be combined may be either same or different.

Alternatively, frequency compounding may be performed with images to be combined obtained by harmonic imaging.

As an alternative embodiment of the optimal sound velocity setting method of the invention, one example is given in which the transmission and reception for sound velocity setting is performed for all images to be combined, a disturbance of reception data is detected, and reception data with the least disturbance for one of positionally-corresponding segment regions of the images to be combined is used for the positionally-corresponding segment regions of all the images to be combined, thereby setting optimal sound velocities of the respective segment regions.

This optimal sound velocity setting method can be applied to both spatial compounding and frequency compounding.

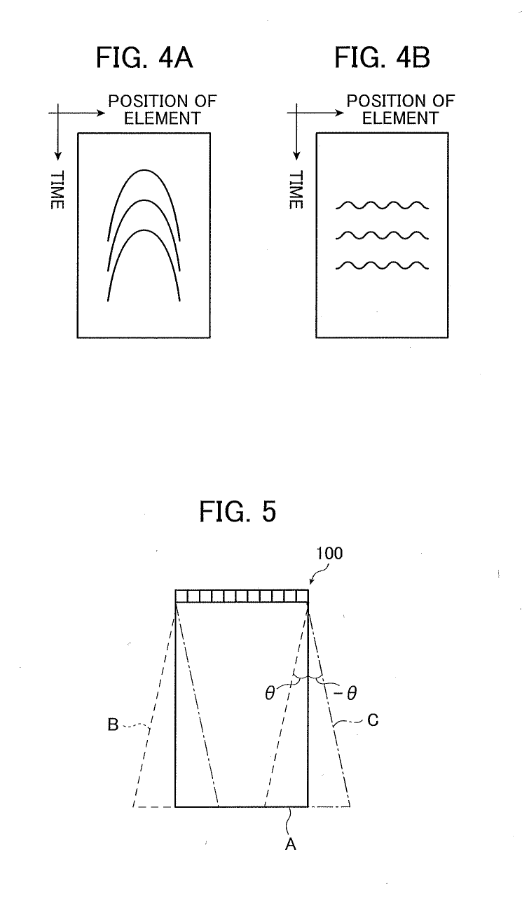

In ordinary ultrasonic echoes, as conceptually shown in FIG. 4A, reception data obtained at each piezoelectric element exhibits a parabolic curve. However, when the wavefront of an ultrasonic wave contains a disturbance, it leads to a disturbance of reception data obtained at each piezoelectric element, as shown in FIG. 4B.

Note that FIG. 4 each show the case where three reflectors are present on a certain ultrasonic beam (sound ray signal to be produced) at equal intervals, where the horizontal axis indicates the azimuth direction, i.e., a position of a piezoelectric element and the vertical axis indicates reception time of an ultrasonic echo.

Reception data having a disturbance is likely to be due to an ultrasonic beam and/or an ultrasonic echo which is negatively affected in the subject. If reception data having a disturbance is used to set optimal sound velocities, it is impossible to set accurate optimal sound velocities.

When reception data whose wavefront has a small disturbance is used to set optimal sound velocities, it becomes possible to consistently set accurate optimal sound velocities for respective segment regions.

One example will be explained with reference to FIGS. 2A and 2B. When optimal sound velocities are set, the transmission circuit 16 and the reception circuit 18 cause the piezoelectric element array 14 to perform the transmission and reception for sound velocity setting for the images to be combined A, B and C.

Reception signals output from the piezoelectric element array 14 are processed by the reception circuit 18 to be reception data, and the reception data is stored in the reception data memory 36.

The ultrasound image producer 50 reads out the reception data from the reception data memory 36, and detects a disturbance of the reception data for each of the segment regions of the images to be combined. Subsequently, the ultrasound image producer 50 compares disturbances of the reception data among positionally-corresponding segment regions of the images to be combined A, B and C, and selects one of the positionally-corresponding segment regions associated with reception data having the least disturbance. This process is performed for each of the segment regions.

Furthermore, the signal processor 20 uses (combines) segment regions thus-selected from the segment regions of the images to be combined to produce reception data (hereinafter referred to as data for sound velocity setting) for the same region as that of the image to be combined A and stores the produced data in the reception data memory 36.

In the following processing, similarly to the foregoing, the sound velocity setting unit 40 supplies the set sound velocities S1 to Sn to the signal processor 20, and the signal processor 20 uses the set sound velocities S1 to Sn to produce sound ray signals, and in response thereto, the ultrasonic wave producer 50 produces B-mode image signals of the data for sound velocity setting. The sound velocity setting unit 40 sets optimal sound velocities of the respective segment regions and supplies the set optimal sound velocities to the signal processor 20 in the same manner as in the above, and whereafter, the signal processor 20 stores the optimal sound velocities of the respective segment regions for the B-mode image signal of the data for sound velocity setting.

When spatial compounding is performed, similarly to the foregoing, the transmission circuit 16 and the reception circuit 18 cause the piezoelectric element array 14 to sequentially perform the transmission and reception for image A, the transmission and reception for image B, and then the transmission and reception for image C.

Reception signals output from the piezoelectric element array 14 through the transmission and reception are processed by the reception circuit 18 to be reception data, and the reception data is supplied to the signal processor 20.

The signal processor 20 having acquired the reception data performs delay correction on reception data resulting from the transmission and reception for the images to be combined A, B and C based on the optimal sound velocities of corresponding segment regions as set for the B-mode image signal of the data for sound velocity setting, thereby producing delay correction data.

Subsequently, similarly to the foregoing, the signal processor 20 adds the produced pieces of delay correction data to produce a sound ray signal, and performs the correction of attenuation and the envelope detection process on the produced sound ray signal, thereby producing the images to be combined A, B and C (B-mode images thereof).

The images to be combined A, B and C undergo raster conversion by the DSC 24 and predetermined image processing by the image processor 26, and the images to be combined A, B and C are combined by the image combining unit 34 to produce a compound image.

The compound image produced by the ultrasound image producer 50 is transmitted to the display controller 28 and then to the display unit 30 to be displayed on the display unit 30.

While the ultrasound diagnostic apparatus, the ultrasound image producing method, and the recording medium of the invention have been described above in detail, the invention is by no means limited to the above embodiments, and various improvements and modifications may be made without departing from the scope and spirit of the invention.

This invention is advantageously applicable to an ultrasound diagnosis used in various kinds of diagnoses in the medical field.

What is claimed is:

1. An ultrasound diagnostic apparatus comprising:
a piezoelectric element array having piezoelectric elements arranged therein, each adapted to transmit ultrasonic waves, receive ultrasonic echoes reflected by a subject, and output reception signals according to received ultrasonic waves;
a controller adapted to control transmission and reception of ultrasonic waves by the piezoelectric element array;
a storage unit adapted to store the reception signals output by the piezoelectric element array;
a sound velocity setting unit adapted to divide the subject into multiple segment regions and set a sound velocity for each of the segment regions with use of the reception signals stored in the storage unit; and
an image producer adapted to produce an ultrasound image by processing the reception signals output by the piezoelectric element array or the reception signals read out from the storage unit based on the sound velocity for each of the segment regions,
wherein the controller has a function of causing the piezoelectric element array to perform transmission and reception for images to be combined to produce images to be combined, the transmission and reception for images to be combined being transmission and reception of ultrasonic waves mutually different in at least one of directions of transmission and reception and central frequencies of ultrasonic waves, and the images to be combined being ultrasound images to be combined;
the image producer has a function of producing the images to be combined with use of the reception signals obtained through the transmission and reception for images to be combined, and combining the produced images to be combined to produce a composite ultrasound image; and
the image producer produces all of the images to be combined based on the sound velocity set for each of the segment regions.

2. The ultrasound diagnostic apparatus according to claim 1, wherein the sound velocity setting unit sets a sound velocity when an instruction to produce the composite ultrasound image is issued.

3. The ultrasound diagnostic apparatus according to claim 1, wherein the image producer produces a main image which is an ultrasound image having a region covering the composite ultrasound image as one of the images to be combined.

4. The ultrasound diagnostic apparatus according to claim 1, wherein the sound velocity setting unit sets sound velocities for all of images to be combined.

5. The ultrasound diagnostic apparatus according to claim 1, wherein the sound velocity setting unit sets sound velocities only for one image to be combined; and
the image producer produces another image to be combined for which no sound velocities are set based on the sound velocities of corresponding segment regions of the one image to be combined for which the sound velocities are set.

6. The ultrasound diagnostic apparatus according to claim 5, wherein the image producer produces a main image which is an ultrasound image having a region covering the composite ultrasound image as one of the images to be combined; and
the one image to be combined for which the sound velocities are set by the sound velocity setting unit is the main image.

7. The ultrasound diagnostic apparatus according to claim 1, wherein when the sound velocity setting unit sets sound velocities, the controller causes the piezoelectric element array to perform transmission and reception of ultrasonic waves for sound velocity setting for all of images to be combined;
distortions of reception signals obtained through transmission and reception of ultrasonic waves for the images to be combined are compared among positionally-corresponding segment regions of the images to be combined, and the sound velocity setting unit sets a sound velocity of a corresponding segment region with use of a reception signal having a least distortion; and
the image producer produces the images to be combined based on the sound velocity set for each of the segment regions of all of the images to be combined.

8. The ultrasound diagnostic apparatus according to claim 1, having a function of selecting one of the images to be combined for which sound velocities are set by the sound velocity setting unit.

9. The ultrasound diagnostic apparatus according to claim 8, wherein the images to be combined are produced from reception signals resulting from transmission and reception of ultrasonic waves for the images to be combined being different in at least directions of transmission and reception of ultrasonic waves; and
the sound velocity setting unit sets sound velocities for an image to be combined being a reference image and sound velocities for another image to be combined resulting from transmission and reception of ultrasonic waves in a direction inclined with respect to a direction of transmission and reception of ultrasonic waves for the reference image by an angle exceeding a predetermined threshold value.

10. The ultrasound diagnostic apparatus according to claim 9, wherein an image to be combined for which no sound velocities are set is produced based on sound velocities of corresponding segment regions of an image to be combined for which the sound velocities are set.

11. The ultrasound diagnostic apparatus according to claim 9, wherein the controller causes the piezoelectric element array to perform transmission and reception of ultrasonic waves for producing a main image which is an ultrasound image having a region covering the composite ultrasound image as the transmission and reception for images to be combined; and
the direction of transmission and reception of ultrasonic waves for the reference image is a direction of transmission and reception of ultrasonic waves for producing the main image.

12. An ultrasound image producing method, comprising the steps of:
causing a piezoelectric element array having piezoelectric elements arranged therein, each adapted to transmit ultrasonic waves, receive ultrasonic echoes reflected by a subject, and output reception signals according to received ultrasonic waves, to perform transmission and reception of ultrasonic waves for a plurality of images to be mutually different in either directions of transmission and reception or central frequencies of ultrasonic waves, or both;
setting a sound velocity for each of segment regions obtained by dividing the subject into multiple segment regions with use of reception signals obtained through transmission and reception of ultrasonic waves for each of the plurality of images, and producing a plurality of images to be combined based on the sound velocity set for each of the segment regions, the images to be combined being ultrasound images to be combined, and
combining the images to be combined to produce a composite ultrasound image.

13. The ultrasound image producing method according to claim 12, wherein the sound velocity is set in response to an instruction to produce the composite ultrasound image.

14. A recording medium having stored therein a program that causes a computer to implement:
a step of causing a piezoelectric element array having piezoelectric elements arranged therein, each adapted to transmit ultrasonic waves, receive an ultrasonic echo reflected by a subject, and output reception signals according to received ultrasonic waves, to perform transmission and reception of ultrasonic waves being mutually different in at least one of directions of transmission and reception and central frequencies of ultrasonic waves so as to produce a plurality of images;
a step of setting a sound velocity for each of segment regions obtained by dividing the subject into multiple segment regions with use of the reception signals obtained through transmission and reception of ultrasonic waves for each of the plurality of images, and producing a plurality of images to be combined being ultrasound images to be combined based on the sound velocity set for each of the segment regions; and
a step of combining the images to be combined to produce a composite ultrasound image.

15. The recording medium according to claim 14, wherein the stored program causes the computer to implement a step of setting the sound velocity prior to the step of performing transmission and reception of ultrasonic waves for the plurality of images.
